(19) United States
(12) Patent Application Publication
(10) Pub. No.: US 2010/0297318 A1
(43) Pub. Date: Nov. 25, 2010

(54) HYBRID MAIZE WITH UNIFORM CONCENTRATION OF ANTHOCYANINS OF EASY EXTRACTION AND PROCESS FOR THE EXTRACTION OF ANTHOCYANINS FROM HYBRID MAIZE

(76) Inventor: Jean Carlo Landivar Bottega, Leme - Sao Paulo (BR)

Correspondence Address: STITES & HARBISON PLLC, 1199 NORTH FAIRFAX STREET, SUITE 900, ALEXANDRIA, VA, 22314, US

(21) Appl. No.: 12/518,443

(22) PCT Filed: Dec. 11, 2006
(86) PCT No.: PCT/BR06/00271

§ 371 Date: Jun. 10, 2009

(51) Int. Cl. A01H 1/02; A01H 1/04; A01H 5/00; A23L 1/10; A23K 1/00

(52) U.S. Cl. 426/462; 800/275; 800/301; 800/320.1

(57) ABSTRACT

HYBRID MAIZE WITH UNIFORM CONCENTRATION OF ANTHOCYANINS, OF EASY EXTRACTION AND PROCESS FOR THE EXTRACTION OF ANTHOCYANINS FROM HYBRID MAIZE, deals with the generation of a hybrid maize with an uniform concentration of anthocyanins and the process for their extraction, providing for the use of anthocyanins and the utilization of maize, without generating rejects in this process, for that purpose, the species in natura containing anthocyanins are properly crossbred with other predefined germplasms, with the proper and adequate characteristics so as to serve as the basal matrix to receive, deposited on its surface, a skin with uniform and easily removable anthocyanins according to the difference of consistence between skin and grain.

### BACKGROUND OF THE INVENTION

1. Field of Invention

The present invention relates to the field of plant breeding, more specifically to hybrid maize breeding.

2. Description of the Prior Art

As it is known by a person of skill in the art, anthocyanins are components used in different types of applications, especially in human health. However, they are much used also as a dying element. In nature, anthocyanins are found in different types of vegetables and fruits. Red and dark colored fruits, such as gooseberry, grape, mulberry, etc.

Anthocyanins are also found in some species of maize, in the grain or in the cob, however, at a much lower concentration than in other types of plants such as the already mentioned fruits.

One of the inconveniences found in the utilization of anthocyanins is their extraction, since they are obtained through the berries of fruits and vegetables. This makes their utilization very difficult, since their extraction is connected to specific periods of the year, in conformance with the fruits season. Also, by extracting the anthocyanins from their berries and peels disables the fruits for other purposes, generating great volumes of rejects and material to be discarded.

In the case of maize, natural species present a very irregular concentration of anthocyanins and the same is concentrated in the skin of the grains (with a soft endosperm), which limits its utilization for the extraction of anthocyanins. The soft endosperm does not allow for the withdrawal of the skin from the grain, leading to the disruption of the grain when it is extracted, a grain which could be commercialized later on. Some natural species of maize present high concentrations of anthocyanins in the cob, which is not edible and, therefore, may not be commercialized for oral use by humans since they are of non-edible origin.

What is needed therefore is a hybrid maize, whose content of anthocyanins is highly uniform and concentrated in the skin of grains, as well as, that this skin is easily removed in contrast with the hardness and quality of the endosperm.

### SUMMARY OF THE INVENTION

The present invention provides a hybrid maize with uniform high concentration of anthocyanins of easy extraction and a process for the extraction of the anthocyanins from the hybrid maize. The invention proposes the generation of a hybrid maize with a uniform concentration of anthocyanins and the process for its extraction, providing for the use of anthocyanins and the utilization of maize, without generating rejects during this process.

In the present invention anthocyanins are introduced in hybrid maize, so that the anthocyanins are homogeneously present in the pericarp of the hybrid maize. Differently from the species found in nature, or even from the currently used hybrid maize, the present invention will allow for the utilization of an efficient process for the extraction of anthocyanins, which is a component that may be used in different types of applications such as: human health, cosmetic, food, pharmaceutical, textile industries, in the production of alcoholic and non-alcoholic beverages and many other applications.

The present invention also provides a process for the extraction of anthocyanins from hybrid maize, that is efficient and that does not damage grains in the process, preserving them and ensuring their posterior utilization, without a significant loss of their nutritional or economic value.

The hybrid maize of this invention contains a uniform content of anthocyanins, with a hard endosperm, able to allow for the withdrawal of anthocyanins from their skin, without damaging grains that will be used. Among other applications for the anthocyanins, they may be used for animal food, since the hard endosperm is orange, very well suited for eggs and chicken, without generating waste.

This hybrid maize with uniform content of anthocyanins is obtained through backcrossing and self-pollination processes among the different species of maize and a specific germplasm, until reaching a homozygote lineage with the desired characteristics, whose coloring in the grain is highly uniform in relation to the natural species.

The process of extraction of anthocyanins enables to withdraw such component and keeping the original characteristics of maize grains, and the maize after being processed may be used in a conventional form, as human or animal food, or in any other way, since its general characteristics are preserved.

The current process does not generate great quantities of materials to be discarded or rejected, while after the extraction of anthocyanins, all the processed material may be reused and used as conventional maize, without any economic loss.

### DETAILED DESCRIPTION OF THE INVENTION

In a preferred embodiment of the current invention, it is used a species of maize, as found in nature, containing anthocyanins. The mentioned anthocyanins are dispersed among the grains, presenting a wide variety of colors, such as white, yellow and, at last, black, which are the grains that may be taken as the source of anthocyanins.

Therefore, this natural species presents a low productivity, due to the color of the endosperm, white or light yellow, which is also soft, that is, it does not present an industrial adaptability.

This species containing a certain content of anthocyanins is properly crossbred with another predefined germplasm, with proper and adequate characteristics so as to serve as the basal matrix. The matrix receives, deposited on its surface, and the skin, with uniform anthocyanins, is easily removable due to the difference of consistence between the skin and the grain.

This basal matrix is the extremely hard endosperm of the germplasm developed by the inventor. Thus, in this first crossing, the resulting materials, are self-pollinated and selected with the previously mentioned specific characteristics, for 10 consecutive cycles, until achieving a homozygote lineage, that may act as male in crossbreeding with a female of germplasm, in which that difference in contrast between the pericarp and the endosperm is intensified to facilitate the withdrawal of the skin from the grain without damaging the grain of the proposed hybrid maize, so that it is obtained an uniform maize plant, containing an uniform content of anthocyanins, generating a vegetal material of hybrid maize that will be used for industrial agricultural development.

According to such characteristics, the stages necessary for this process are the following:

Firstly, it is used a source of anthocyanins, which may be the species referred to as found in nature as mentioned above, to be the donor of anthocyanins. This species is sown in lines of five meters of length, together with the germplasm developed by the inventor (this germplasm is properly organized into heterotic groups as per molecular marker through SSRs microsatellites) being used as female, also in lines of five meters of length, taking care so that the plantation date ensures the synchronization of the male flowering of the species with the female flowering of the germplasm. At the time of flowering, the pollen of the species that acts as male is taken to the receptive stigmas of the germplasm, properly protected by paper bags before their emergence, to ensure the desired specific directed crossbreeding, and pollination is then promoted. The plants of the species that serve as donors of pollen must be carefully chosen, by selecting only those that present the desirable characteristics similar to those mentioned below (characteristics of the plant), which are those the germplasm has and which are those that are desired for this improvement. Soon after, the stigmas still remain protected not to allow for any other type of pollen to interfere.

The characteristics of the specific germplasm, that receives the pollen from the species, to become able for industrialization are comprised into two basic categories to be analyzed and are visualized as particular characteristics of the grain and particular characteristics of the plant:

Within these specifications, the characteristics of the grain are the following:
1—Hard endosperm.
2—Good quality of grain.
3—Resistance to main tropical diseases of the grain.
4—Resistance to grain breaking.
5—Resistance to grain speckling by the rain.
6—Smooth pericarp.
7—Orange endosperm.
8—Long grains.
9—Bright pericarp and resistant to stubbing.
10—Grains with a smaller marking characteristic of the type indented.
11—Pericarp with capacity to add color to the region of the embryo.
12—Easy of threshing below 30% of humidity.

Following the same principle of grain characteristics, the plant must also present specific characteristics, which are the following:
1—Good stuffing of the ears.
2—Resistance to lodging.
3—Resistance to main tropical diseases of the leaves.
4—Resistance to breaking.
5—Resistance to stub of the leaves by the wind.
6—Intense green color of the leaves.
7—Low height of plant.
8—Thicker stalks.
9—Low plant height vs. ear height ratio.
10—Fast capacity to loose water after the physiological maturity of the plant.
11—Capacity of the plant to stand straight for mechanical harvesting.
12—Precocity to flowering.
13—Precocity to harvesting.
14—Coincidence between male and female flowering.
15—Fast emergence,
16—Plant vigor.
17—Good viability of pollen in tropical environment.
18—Wind stress Tolerance.
19—High total evaporation tolerance to stress.
20—Stress tolerance due to lack of water.

In order to obtain the characteristics above, after crossbreeding, it is necessary to wait for the filling of the grain, its physiological maturity and, at the time of harvesting, the best ears are selected by choosing only those from the lines of the germplasm. The natural species, as of this time, is discarded, that is, practically at the start of the improvement work, since it already contributes with its pollen, and the seeds collected from the ears corresponding to the germplasm will be used.

At the time of harvesting, it is very important to select only the ears that present grains with the best colors, aiming at the possibility of a better transfer of anthocyanins, by selecting the grains of color black or intense red, since a natural species may not have consistent typical characteristics and the normal case is that they present a wide genetic variability, especially in the color of the grain.

The following stage is undertaken after harvesting, in which the ears are dried in an ears drier, at a temperature of approximately 38° C. and with an intense flow of air. The drying of ears is important to enable to evaluate which grains present the best color tones closer to black, the color that is of interest.

Thus, the selection of more colored ears is done and, carefully, selecting only the black or intense red grains, which are separated into packages taken from each ear, to be sown again in differentiated lines. All grains of other colors must be necessarily discarded so as not to hinder the improvement that starts as of now from this initial material.

The following stage starts with the above-mentioned packages containing the seeds of the first crossing (defined as F1) being sown in individual lines, properly identified with a number, for each row until completing five meters of length. In case there are exceeding seeds in each row one must select the best seeds, by giving again priority in the selection to those of a darker color.

Again, it is necessary to sow on the side of each row of F1, a row for the initial germplasm of the inventor, taking care so that the flowering of both lineages is synchronized for the new crossbreeding. The F1 act as male and the germplasm as female, again. At the time of flowering, the pollen of the F1, which acts as male, is taken to the receptive stigmas of germplasm, being properly protected by paper bags before their emergence to ensure the desired specific directed crossbreeding, and pollination is effected. The plants from F1 that will serve as donors of pollen must be carefully chosen, by selecting only those that present the desirable characteristics, which are those the germplasm has and that are intended for the improvement. Thus, after pollination, the stigmas still remain protected to avoid that any other type of pollen to interfere.

At the following stage, after crossbreeding, one must wait for the filling of the grain, its physiological maturity and, at the time of harvesting, the best ears are selected, only from the lines of the germplasm. The row of the F1, as of this time, is discarded, since it already contributes with its pollen, and the seeds collected from the ears corresponding to the germplasm are defined as BCI. At this stage, it is very important that at the time of harvesting, only the BC1 ears that present a greater quantity of grains with the best colors are selected, aiming at the possibility of a better transfer of anthocyanins, since the BC1 start to show certain selectable characteristics, however, they do not present yet a consistence in the desired characteristics. The normal is that they present a wide genetic variability, especially in the color of the grain. In any case, it is necessary to select the parameters mentioned above by emphasizing the black color grains, and discarding all other ones, including the red or reddish grains, that could be selected in the harvesting of the previous one. At this stage, the selection criteria are more specific. After harvesting, the selected ears are dried in an ears drier, at a temperature of approximately 38° C. and with an intense flow of air. This drying is important to evaluate which grains presents the best tones closer to black. After that, it is necessary to select the more colored ears and, carefully. It should be selected only the black or intense red grains, which are separated into packages derived from each properly identified BC1 ear. All grains of other colors are necessarily discarded so as not to hinder the improvement, whose selection criteria are intensified in the next stages.

Thus, the packages containing the BC1 seeds are sown in individual lines, properly identified with numbers, each row at a time until completing five meters of length. If there are exceeding seeds in each row, the best seeds must be selected, by giving priority again in the selection to the darker colored seeds. At the time of flowering, all BC1 seeds have their pollen taken to the receptive stigmas of the same plants, being then self-pollinated. The stigmas must be properly protected by paper bags before the emergence of the same, to ensure the desired specific directed crossbreeding. The plants of the BC1 to be self-pollinated must be carefully chosen, so that those that present the desirable characteristics next to those already mentioned, which are those the germplasm already has, and which we intend to obtain, thus intensifying the selection criteria.

After pollination, the stigmas still remain protected to avoid that other types of pollen interfere.

Therefore, it is necessary to wait for the filling of the grain, its physiological maturity and then start the strict selection of leaf diseases, while the plants that present diseases after being self-pollinated, must be equally discarded. At the time of harvesting, the best ears from the plants with the selected diseases are selected; the remaining ones are discarded, no matter what type of grain they present. At this stage it is extremely important to collect only the ears that present a greater quantity of grains with an intense black color, by selecting the transfer of anthocyanins, which are denominated S1, whose grains start to show the selectable characteristics, which may be transferred to the following cycles. However, they do not present yet the consistence in their desired characteristics. The normal is that they present a wide genetic variability, especially in the color of the grain. At this stage, the selection criteria become even more rigid, being inflexible as regards black grains.

After harvesting, the ears are dried in an ears drier, at temperature of approximately 38° C. and with an intense flow of air. The drying is important so that it is actually evaluated which grains present the best tones and closer to black.

Thus, a selection of the more colored ears is done and one must carefully select only the black or intense red grains, and these are separated into packages derived from each identified S1 ear. All grains of other colors must be necessarily discarded.

After this stage, the packages containing the S1 seeds are sown to start with the endogamic depression until an advanced homocygosis of the lineages is achieved. Also in individual lines, properly identified with their own number, for each row until completing five meters of length. In case there are exceeding seeds for each row one must select the best seeds, by giving priority again in the selection to the darker colored seeds.

At the time of flowering, all S1 seeds and their pollen are taken to the receptive stigmas of the same plants, being then self-pollinated. The stigmas must be properly protected by paper bags before the emergence of the same to ensure the desired specific directed crossbreeding. The plants of the S1 to be self-pollinated must be carefully selected, especially those that present the desired characteristics. After pollination, the stigmas remain protected not to allow any other type of pollen to interfere.

Therefore, it is necessary to wait for the filling of the grain, its physiological maturity. The strict selection of leaf diseases starts, and plants that present diseases after being self-pollinated, are discarded. At the time of harvesting, the best ears from the plants that will be used in the selection of diseases are selected, the remaining ones are discarded, no matter what type of grain they present.

At this stage it is critically important to collect only the ears that present a greater quantity of grains with an intense black color, by selecting a transfer of anthocyanins. These grains are defined as S2, which start to show selectable characteristics that may be transferred to the following cycles, since the number of black grains increases in the ears, and more than half of these ears have completely black grains.

After harvesting, the ears are dried in an ears drier, at a temperature of approximately 38° C. of and with an intense flow of air. It is necessary to select grains with the best tones and closer to black. After the selection of more colored ears has been done, carefully, it is selected only the black or intense red grains, them being separated into packages derived from each identified S2 ear. All grains of other colors are necessarily discarded.

At the following stage, the packages containing the S2 seeds are sown in individual lines, properly identified with their own number, for each row until completing five meters of length. In case there are exceeding seeds for each row one must select the best seeds, by giving priority again in the selection to the darker colored seeds.

At the time of flowering, all S2 seeds, whose pollen is taken to the receptive stigmas of the same plants, are self-pollinated. The stigmas must be properly protected by paper bags before the emergence of the same to ensure the desired specific directed crossbreeding. The plants of the S2 to be self-pollinated must be carefully chosen, to present the desirable characteristics, so that the characteristics are transferred to the following generation. Thus after that, the stigmas still remain protected not to allow that any other type of pollen to interfere.

After crossbreeding, it is necessary to wait for the filling of the grain, its physiological maturity, and then start with the strict selection of leaf diseases, while the plants that present diseases after being self-pollinated, are discarded. At the time of harvesting, the best ears are selected from plants that have the selection of diseases; the remaining ones are discarded, no matter what type of grain they present. At this stage it is critically important to collect only the ears that present a greater quantity of grains with an intense black color, acquiring the transfer of anthocyanins, these grains being defined as S3, which show selectable characteristics that may be transferred to the following cycles. At this time, the quantity of black grains of each ear start to increase, and it may already be found more than half of the ear with completely black grains. While here the selection criteria are more intense, however, black grains are found in greater quantity and it is noticeable the diminution in the size of the ears due to the homocygosis that is accentuated.

After harvesting, the ears are dried in a ears drier, at a temperature of 38° C. and with an intense flow of air. It is important to identify the grains that present the best tones and closer to black.

Therefore, it is necessary to select the more colored ears and, carefully, only the black grains are separated into packages derived from each identified S3 ear. All grains of other colors are necessarily discarded. Due to the fact that the biosynthesis of anthocyanins in maize is controlled by multiple regulator genes and induced by the development of many environmental factors, involving more then 20 loci and the expression of many genes in the distribution of the pigment in the tissues, and the origin of the 2n pericarp derives from the female; at the following stage, crossbreeding tests are performed to separate the improvement of lineages into two specific groups, which is necessary to turn the lineages with anthocyanins into homozygote ones:

In group 1: the male lineages that did not blacken the female's seeds at the time of crossbreeding in the production fields, but in the harvesting of grains at the level of the producer's field and which acquired anthocyanins for industrialization.

In Group 2: the male lineages whose female seeds have been blackened at the time of crossbreeding in the seeds production fields, and also the harvesting of the grains at the level of the producer's field and that acquired anthocyanins for industrialization.

Therefore, the first crossbreeding tests are started with the 2 female of the heterotic group E (of the germplasm), to separate the lineages that carry anthocyanins in the formation of the two specific groups mentioned above. Again, the packages containing the S3 seeds are sown in individual lines, properly identified with their own number, for each row until completing five meters of length. In case there are exceeding seeds for each row it is necessary to select the best seeds, by giving priority again in the selection to the darker colored seeds. It is also sown on the side with same length, the female plant for each male S3 plant to perform crossbreeding.

At the time of flowering, all S3 seeds and their pollen are taken to the receptive stigmas of the same plants, them being self-pollinated and marked with a number. The stigmas both of the S3 seeds and of the female plants of group E must be properly protected by paper bags before the emergence of the same to ensure the desired specific directed crossbreeding. The plants of S3 seeds to be self-pollinated must be carefully chosen, especially those that present the desired characteristics, ensuring that the characteristics will be transmitted to the next generation. And after pollination, the stigmas must still remain protected not to allow any that other type of pollen to interfere. At the same time that each S3 plant is self-pollinated and marked with its number, it is necessary to proceed with crossbreeding with the two female of group E, marking also the code of the S3 plant to identify the origin of the male with anthocyanins.

After the crossbreeding, it is necessary to wait for the filling of the grain, its physiological maturity, and now it is started a strict selection of leaf diseases within the S3 group, and the plants that present diseases after being self-pollinated must be equally discarded. At the time of harvesting, the best ears from the plants that have gone through the selection of diseases are selected, and the remaining ones must be discarded, no matter what type of grain they present.

At this stage it is critically important to collect only the ears that present a greater quantity of grains with an intense black color, acquiring the transfer of anthocyanins, the grains derived from the harvesting which show selectable characteristics that may be transferred to the following cycles being denominated S4. At this time, there starts to appear completely black ears and the genetic variability in the color of mixed grains start to diminish. The completely identified female plants must also be collected. After harvesting, the ears are dried in a ears drier, at a temperature of 38° C. and with an intense flow of air, an optimal drying being important so it may be possible to actually evaluate which grains present the best tones and closer to black.

After that, the more colored ears are selected and, carefully, only those with black grains are selected, which are separated into packages derived from each identified S4 ear. All grains of other colors must be necessarily discarded.

At the following stage it is necessary to start by planting the crossbreeds of the female of heterotic group E, in 8 lines of 5 meters of length but separated into two groups mentioned in the previous stage, by selecting by color the seeds collected from the female plant, and the remaining seeds of normal color shall belong to group 1, and to group 2 those that remain black only due to the pollen of the S4 plant at the time of crossbreeding. The production test must be proceeded with until harvesting to observe which of the S4 plants have heterosis with the female ones. These testes are known as precocious production tests.

The packages containing the S4 seeds are sown in individual lines, properly identified with their related number, for each row until completing five meters of length, them being divided into groups 1 and 2 according to the information on color taken from the seeds of the female plant. At the time of flowering, all S4 plants and their pollen are taken to the receptive stigmas of the same plants, being self-pollinated and marked with a number. The stigmas of the S4 plants must be properly protected by paper bags before the emergence of the same. The plants of the S4 lineage to be self-pollinated must be carefully chosen, especially those that present the desired characteristics. Thus after that, the stigmas still remain protected not to allow any other type of pollen to interfere.

After crossbreeding, it is necessary to wait for the filling of the grain, its physiological maturity, and it is started the strict selection of leaf diseases within the S4 group, while the plants that present diseases after being self-pollinated must be equally discarded. At the time of harvesting, the best ears from the plants that have gone through the selection of diseases are selected, while the remaining ones must be discarded, no matter what type of grain they present. At this stage it is critically important to collect only the ears that present all grains with an intense black color, acquiring the transfer of the anthocyanins, which are defined as S5. After harvesting, the ears are dried in an ears drier, at a temperature of 38° C. and with an intense flow of air, such task being important to identify which grains present the best tones of black. These are separated into packages derived from each identified S5 ear.

At the following stage, it is started the harvesting of the crossbreeds of the female plants of heterotic group E, that were sown in the previous stage, and by identifying the best 10% productions of grain and, with this information, they are selected as S5 male. The sowing of the packages containing the S5 seeds must continue for each row until completing five meters of length, them being divided into groups 1 and 2 according to the information on color taken from the seeds of the female plant. At the time of flowering, all S5 and their pollen are taken to the receptive stigmas of the same plants, them being self-pollinated and marked with their related number. The stigmas of the S5 must be properly protected by paper bags before the emergence of the same. The plants of the S5 group to be self-pollinated must be carefully chosen, especially those that present the desirable characteristics so that these characteristics are transmitted to the following generation. Soon after that, the stigmas must still remain protected not to allow any that other type of pollen to interfere.

After the crossbreeding, it is necessary to wait for the filling of the grain, its physiological maturity, and it is started a strict selection of leaf diseases within the S5 group, and the plants that present diseases after having been self-pollinated must be equally discarded. At the time of harvesting, the best ears from the plants that have gone through the selection of diseases are selected, while the remaining ones are discarded, no matter what type of grain they present. Again at this point it is critically important to collect only the ears that present all grains with an intense black color, since these are the plants that have assimilated the transfer of anthocyanins, these grains being defined as S6. After harvesting, the ears are dried in an ears drier, at a temperature of 38° C. and with an intense flow of air, an optimal drying being important so it is enabled to actually evaluate which grains present the best dark colored tones. Such grains are separated into packages derived from each differentiated S6 ear.

At the following stage, it is necessary to continue to sow the packages containing the S6 seeds in each row until completing five meters of length, keeping them in groups 1 and 2 according to the information on color taken from the seeds of the female plant. At the time of flowering, all S6 and their pollen are taken to the receptive stigmas of the same plants, them being self-pollinated and marked with their related number. The stigmas of the S6 must be properly protected by paper bags before the emergence of the same. The plants of the S6 to be self-pollinated must be carefully chosen, especially those that present the desirable characteristics. Soon after, the stigmas must still remain protected not to allow any that other type of pollen to interfere.

After the crossbreeding it is necessary to wait for the filling of the grain, its physiological maturity, and it is started again the process of strict selection of leaf diseases within the S6, while the plants that present diseases after having been self-pollinated must be equally discarded. At the time of harvesting, the best ears from the plants that have gone through the selection of diseases are selected, while the remaining ones are discarded, no matter what type of grain they present.

Again, at this point it is critically important to collect only the ears that present all grains with an intense black color, which are those that have assimilated the transfer of anthocyanins, which are properly denominated S7. After harvesting, the ears are dried in an ears drier, at a temperature of 38° C. and with an intense flow of air, this procedure being important so it is enabled to actually evaluate which grains present the best colored dark tones. These grains are separated into packages derived from each identified S7 ear.

At the following stage, each S7 package goes through an increment in the quantity of seed in isolated loci, according to the SSRs isolation and molecular marking norms to group the lines within the heterotic groups of male plants, discarding those that have a spacing coefficient greater then 0.77, thus becoming a lineage.

At the following stage, each lineage that presents a good precocious production test, starts to be tested with all female plants of the defined germplasm and form hybrids that, at the level of production, present a completely black color in its grains, besides two other different types at the level of seeds, which have a normal color and which have seeds of black color. The hybrids are completely different from the species as found in nature.

Therefore, these hybrids comply with the desired characteristics of the grain and the desired characteristics of the plant as already previously defined. The obtained plant, presents completely black grains due to the anthocyanins of the pericarp, and commercial production is possible, according to the seeds production norms.

The plantation of these seeds may be undertaken with due assistance by using the same conventional planting equipment. The harvesting of the hybrid maize is done by means of a conventional harvesting machine with humidity from 25% to 30% where the grains are separated from the rest of the plant, such as: stem, leaves, straw and cobs, the grains being separated and forwarded to processing.

At this stage it is started the process for the extraction of anthocyanins, and its first stage comprises the cleaning of the harvested maize, where the impurities that have not been separated by the harvesting machine are removed by the pre-cleaning and cleaning machines, proceeding to the following stage, where, they are forwarded to a specific machine for the withdrawal of the skin from their grains. At this other stage all grains' skin withdrawal is processed in another machine, this separation being easily done due to the difference of hardness between skin and the grains and the skin is properly processed, turning the collected material into a composite in the form of powder that is later on processed chemically for the extraction of anthocyanins.

At the following stage of the current process the powdered material is processed chemically so that the anthocyanins are properly isolated. The anthocyanins are separated and processed in the form of powder; however, their form of presentation may be different, according to the need of their use.

At the following stage, the grains without skin are properly dried, in a conventional drier, until they reach 13% of humidity, especially for their storage in bags or they may be forwarded to storage silos, and they may be normally commercialized both as human food or as animal food, since their characteristics are preserved, by keeping both their nutritional value, and their economic value.

Finally, the anthocyanins may be commercialized as pure, in different forms of presentation, that is, in capsules, in powder, liquid, granules, etc., in conformance with the need of their use.

1-7. (canceled)

8. A process for producing a hybrid maize having the characteristics of a hard endosperm and a uniform high content of anthocyanins, the process comprising the steps of:
(a) crossbreeding a natural maize species with a high content of anthocyanins with a predefined germplasm to obtain a crossbred material;
(b) self-pollinating and selecting the crossbred material with said characteristics for at least ten consecutive cycles, and achieving a homozygote lineage that is capable of acting as a male in crossbreeding with female germplasm, and
(c) obtaining a uniform maize plant, containing a uniform high content of anthocyanins, and generating a vegetal material of hybrid maize suitable for industrial and agricultural uses.

9. A process for producing a hybrid maize having the characteristics of a hard endosperm and uniform high content of anthocyanins, the process comprising the steps of:
(a) crossbreeding a natural maize species with a predefined female germplasm, said predefined female germplasm organized into heterotic groups by molecular markers through SSRs microsatellites; said crossbreeding arranged so that flowering is synchronized enabling pollen from the natural species to be directed to stigmas of the predefined female germplasm, and obtaining a crossbred material;
(b) selecting the obtained crossbred material having said characteristics;
(c) protecting the material;
(d) promoting pollination of the selected obtained crossbred material;
(e) waiting for the filling and maturity of grain, resulting from the pollination, and obtaining ears of filled and mature grains;
(f) harvesting selected ears with said characteristics and discarding the non-selected ears;
(g) drying the selected ears;
(h) selecting and separating dark red or black colored grains from the selected ears with said characteristics; and
(i) repeating steps (a) to (h) nine times to complete a total of ten cycles so as to obtain the hybrid maize with hard endosperm and uniform high content of anthocyanins.

10. The process for producing a hybrid maize according to claim 9 wherein the drying step is conducted in a suitable drier at a temperature of 38° C.

11. The process for producing a hybrid maize according to claim 9, further comprising performing a selection step that hinders the development of diseases of said hybrid maize, said selection step starting no earlier than the third of the ten cycles.

12. The process for producing a hybrid maize according to claim 9, wherein in the last cycle, each lineage presenting a good production precocious test is then tested with all female specimen of the predefined female germplasm, producing completely black hybrids grains, normal color seeds and black colored seeds.

13. A hybrid maize produced according to the process of claim 9, wherein the grains of the hybrid maize have the physiological and morphological characteristics of: (1) hard endosperm, (2) good quality of grain, (3) resistance to main tropical diseases of the grain, (4) resistance to grain breaking, (5) resistance to grain speckling by rain, (6) smooth pericarp, (7) orange endosperm, (8) long grains, (9) bright pericarp and resistant to stubbing, (10) grains with a smaller marking characteristic of the type indented, (11) pericarp with capacity to add color to the region of the embryo, and (12) easy of threshing below 30% of humidity.

14. A hybrid maize produced according to the process of claim 9, wherein the hybrid maize plant has the physiological and morphological characteristics of: (1) good stuffing of the ears, (2) resistance to lodging, (3) resistance to main tropical diseases of the leaves, (4) resistance to breaking, (5) resistance to stub of the leaves by the wind, (6) intense green color of the leaves, (7) low height of plant, (8) thicker stalks, (9) low plant height vs. ear height ratio, (10) fast capacity to loose water after the physiological maturity of the plant, (11) capacity of the plant to stand straight for mechanical harvesting, (12) precocity to flowering, (13) precocity to harvesting, (14) coincidence between male and female flowering, (15) fast emergence, (16) plant vigor, (17) good viability of pollen in tropical environment, (18) wind stress tolerance, (19) high total evaporation tolerance to stress; and (20) stress tolerance due to lack of water.

15. A seed of the hybrid maize produced according to the process of claim 9.

16. A hybrid maize plant, or a part thereof, produced by growing the seed according to claim 15.

17. Pollen of the hybrid maize plant according to claim 16.

18. An ovule or ovules of the hybrid maize plant according to claim 16.

19. A tissue culture of regenerable cells produced from the hybrid maize plant according to claim 16.

20. Protoplasts or callus produced from the tissue culture according to claim 19.

21. The tissue culture according to claim 19, wherein the regenerable cells of the tissue culture are produced from protoplasts or from tissue of a plant part selected from the group consisting of: immature embryo, embryo, meristematic cells, immature tassels, microspores, pollen, root, root tip, anther, silk, leaf, flower, kernel, ear, cob, husk and stalk.

22. A process for the extraction of anthocyanins from the hybrid maize produced according to the process of claim 9, the process comprising the steps of:
(a) harvesting the obtained hybrid maize, containing grains with a humidity ranging from 25% to 30%;
(b) separating the grains of the hybrid maize from the plant;
(c) cleaning the separated harvested grains;
(d) withdrawing the skin from the grains;
(e) processing the withdrawn skin into a powder composite;
(f) chemically processing the powder composite material, isolating the anthocyanins, from the composite;
(g) reserving the isolated anthocyanins, and
(h) drying the grains without skin.

23. The process according to claim 22, wherein the grains without skin are dried to a humidity of 13%.

24. The process according to claim 22, wherein the step of cleaning the separated harvested grains is performed by pre-cleaning or cleaning machines.

25. The process according to claim 22, wherein the reserved isolated anthocyanins are in the form of powder or any other suitable form according to needs or use.

26. The process according to claim 22, wherein the step of drying the grains without skin is performed in a conventional drier.

27. The process according to claim 22, wherein the dried grains without skin may be stored or commercialized as a human food or as an animal food.
